(19) United States
(12) Patent Application Publication
(10) Pub. No.: US 2019/0167506 A1
(43) Pub. Date: Jun. 6, 2019

(54) NECK TRACTION APPARATUS HAVING TRACTION ASSISTING DEVICE

(71) Applicant: CSE CO., LTD., Siheung-si (KR)

(72) Inventor: Ei Jae SUNG, Bucheon-si (KR)

(73) Assignee: CSE CO., LTD, Siheung-si (KR)

(21) Appl. No.: 16/313,248

(22) PCT Filed: May 15, 2017
(86) PCT No.: PCT/KR2017/005007

§ 371 Date: Dec. 26, 2018

(30) Foreign Application Priority Data
Jun. 27, 2016 (KR) 20-2016-0003675
Dec. 16, 2016 (KR) 10-2016-0172453
Feb. 16, 2017 (KR) 10-2017-0020822

(51) Int. Cl. A61H 1/02; A61H 9/00; A61F 5/055; A61F 5/048

(52) U.S. Cl. CPC A61H 1/0296; A61H 9/0078; A61F 5/055; A61F 5/048; A61H 2203/0456; A61H 2205/04; A61H 2201/5035; A61H 2201/5007; A61H 2201/1609; A61H 2201/0103; A61H 2201/5071

(57) ABSTRACT

Provided is a neck traction apparatus for providing cervical correction by stretching the neck. More particularly, the neck traction apparatus has a traction assisting device. The apparatus includes an integral skin foam (ISF) type traction assisting device is provided so as to eliminate discomfort caused by skin contact and to match personal characteristics of a user by extending the stretching length; includes a battery so as to provide portability for use; allows the battery to be physically and electrically isolated when external power is not applied so as to minimize discharging of the battery while in standby; and has a traction body of which inflation and deflation are operated in an automatic mode so as to obtain an optimal effect of the cervical correction and prevent a safety accident from occurring.

### TECHNICAL FIELD

The present invention relates generally to a neck traction apparatus for providing cervical correction by stretching the neck. More particularly, the present invention relates to a neck traction apparatus having a traction assisting device, wherein the apparatus includes an integral skin foam (ISF) type traction assisting device that is provided so as to eliminate discomfort caused by skin contact and to match personal characteristics of a user by extending the stretching length; includes a battery so as to provide portability for use; allows the battery to be physically and electrically isolated when external power is not applied so as to minimize discharging of the battery while in standby; and has a traction body of which inflation and deflation are operated in an automatic mode so as to obtain an optimal effect of the cervical correction and prevent a safety accident from occurring.

### BACKGROUND ART

In general, the cervical vertebrae are the series of bones which are stacked on top of each other with the intervertebral discs (intervertebral fibrocartilages) located between each bone to provide cushioning for body loads and help absorb shocks.

The cervical vertebrae are surrounded by the discs, which are attached between each vertebra and surround and protect the nucleus pulposus, and by fibrous tissues known as ligaments and thus do not easily slip out of place. However, when the body's center of gravity is displaced due to external shock or stimulus, the discs may be crushed and slip out of place.

Such a disc slipped out of place may press on the nearby nerve, causing pain and other symptoms related to the neck, shoulders, arms, fingers, and the like. When severe pain is produced, this may lead to a serious condition that can impair a person's normal life.

Drug treatment, physical treatment, traction treatment, and surgical treatment are mainly used as methods for treating cervical disc disease.

As a conventional technique for a traction apparatus used for cervical traction treatment, Korean Patent No. 10-0483176 disclosed a neck traction apparatus which includes a pad, an inflatable tube positioned at each upper and lower side of the pad, and an air pump for injecting air into the tubes.

Such a neck traction apparatus corrects the cervical vertebrae by repeatedly performing the processes of stretching and releasing the neck according to the inflation and deflation of the tubes due to the injection and discharge of air.

The above-mentioned Korean Patent No. 10-0483176 is problematic in that because the tubes are connected to the opposite sides of the pad in a secured manner and the tubes have a fixed inflation length, it may be difficult to match to the neck length differing for each user and to obtain the desired stretching force differing for each user.

Additionally, the above-mentioned Korean Patent No. 10-0483176 is still problematic in that it may be inconvenient for the user to inject air for stretching by using the air pump, whereby the stretching and releasing of the neck are performed arbitrarily by the user thus leading to a reduction in effect of the cervical correction, and causing a safety accident due to over-inflation of the tubes.

### DISCLOSURE

### Technical Problem

Accordingly, the present invention has been made keeping in mind the above problems occurring in the neck traction apparatus in the related art, and an objective of the present invention is to further provide a traction assisting device which a user may select, whereby even when each user differs in neck length, cervical traction treatment is possible, and the user's neck is stretched with the desired force suitable for each user, thus improving effectiveness and satisfaction of cervical traction treatment.

Another objective of the present invention is to provide a battery for power supply so as to provide portability for use, and further provide a controller having an isolation circuit part, whereby when external power is not applied, the battery is physically and electrically isolated, thus preventing the battery from being consumed while in standby and thus minimizing the discharging of the battery.

Still another objective of the present invention is to automatically repeatedly perform stretching and releasing of the neck when the user sets a controller to an automatic mode without manually manipulating stretching and releasing through the controller, thus obtaining the optimal effect of the cervical correction while preventing a safety accident from occurring by preventing the neck from being stretched with excessive force.

### Technical Solution

In order to accomplish the above objectives, the present invention provides a neck traction apparatus having a traction assisting device, the apparatus including:

a main body having an open front portion and wrapping around a neck of a user;

a traction body connected to an upper portion of the main body and having an open front portion, the traction body wrapping around the neck of the user, the traction body being configured to inflate to stretch the neck;

a vibration unit embedded in the main body to vibrate the main body;

an air injection unit embedded in the main body and injecting air into the traction body to inflate the traction body;

a traction assisting device placed on an upper portion of the traction body and wrapping the neck.

The apparatus may further include a controller including a battery supplying power for driving,

an isolation circuit part providing connection or disconnection with external power for charging the battery,

a charging control part managing charging and discharging of the battery, and

a central control part controlling the charging control part, the vibration unit, and the air injection unit.

Furthermore, the controller may operate the air injection unit in an automatic mode in which a process of injecting air into the traction body for 20 seconds, maintaining stretching for 20 seconds, and then releasing by discharging the air from the traction body for 15 seconds is repeated for 15 minutes.

### Advantageous Effects

According to the neck traction apparatus having a traction assisting device according to the present invention having the above-described configuration, the traction assisting device is selectively used depending on the neck length and the desired stretching force for each user, thus making it possible to achieve improved effectiveness and satisfaction of cervical traction treatment.

Furthermore, it is possible to improve portability and ease of use by supplying power through the battery, and to minimize the discharging of the battery through the controller having the isolation circuit part.

Furthermore, according to the neck traction apparatus having the traction assisting device, it is possible to perform stretching and releasing in the automatic mode, thus eliminating the inconvenience of manual manipulation and to maximize the effect of the cervical correction, while preventing a safety accident from occurring by preventing the neck from being stretched with excessive force or long-term operation. Accordingly, the apparatus is significantly useful invention for industrial development.

### DESCRIPTION OF DRAWINGS

FIGS. 1a and 1b are perspective views showing a neck traction apparatus having a traction assisting device according to an embodiment of the present invention.

FIG. 2 is a block diagram showing a functional structure of a controller.

FIGS. 3a and 3b are circuit diagrams schematically showing a circuit structure of the controller.

FIG. 4 is an exploded view showing a main body and a traction body from which an outer cover is removed.

FIG. 5 is a view showing an internal structural of the present invention.

FIG. 6 is a plan view, a rear view, and a side view showing the main body and the traction body.

FIG. 7 is a view showing a traction assisting device.

FIGS. 8 and 9 are flow charts showing traction and inflation processes of the traction body.

### DESCRIPTION OF THE REFERENCE NUMERALS IN THE DRAWINGS

N: present apparatus (neck traction apparatus)
1: main body
11: support body
13: first mounting portion
15: second mounting portion
2: traction body
21: tube
3: vibration unit
4: air injection unit
5: controller
51: central control part
52: isolation circuit part
521: relay circuit
53: charging control part
531: charging circuit
532: discharging circuit
6: traction assisting device
61: seat portion
62: first side portion
63: second side portion
64: rear side portion
7: fastening member
A: adaptor
B: battery

### BEST MODE

Hereinbelow, a neck traction apparatus having a traction assisting device according to the present invention will be described in detail with reference to the drawings.

Prior to describing the invention in more detail,

The present invention will now be described in detail based on aspects (or embodiments). The present invention may, however, be embodied in many different forms and should not be construed as being limited to only the embodiments set forth herein, but should be construed as covering modifications, equivalents or alternatives falling within ideas and technical scopes of the present invention.

In the figures, like reference numerals, particularly, tens and units, or reference numerals having like tens, units, and letters refer to like elements having like functions throughout, and unless the context clearly indicates otherwise, elements referred to by reference numerals of the drawings should be understood based on this standard.

Also, for convenience of understanding of the elements, in the figures, sizes or thicknesses may be exaggerated to be large (or thick), may be expressed to be small (or thin) or may be simplified for clarity of illustration, but due to this, the protective scope of the present invention should not be interpreted narrowly.

The terminology used herein is for the purpose of describing particular aspects (or embodiments) only and is not intended to be limiting of the present invention. As used herein, the singular forms are intended to include the plural forms as well, unless the context clearly indicates otherwise. It will be further understood that the terms “comprises”, “comprising”, “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms including technical and scientific terms used herein have the same meaning as commonly understood by one of ordinary skill in the art to which the present invention belongs. It will be further understood that terms used herein should be interpreted as having a meaning that is consistent with their meaning in the context of this specification and the relevant art and will not be interpreted in an idealized or overly formal sense unless expressly so defined herein.

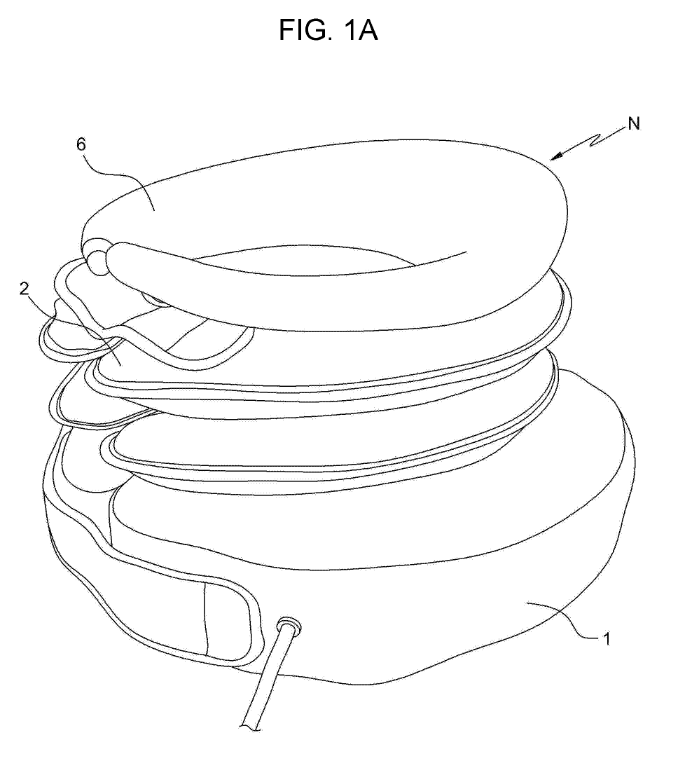

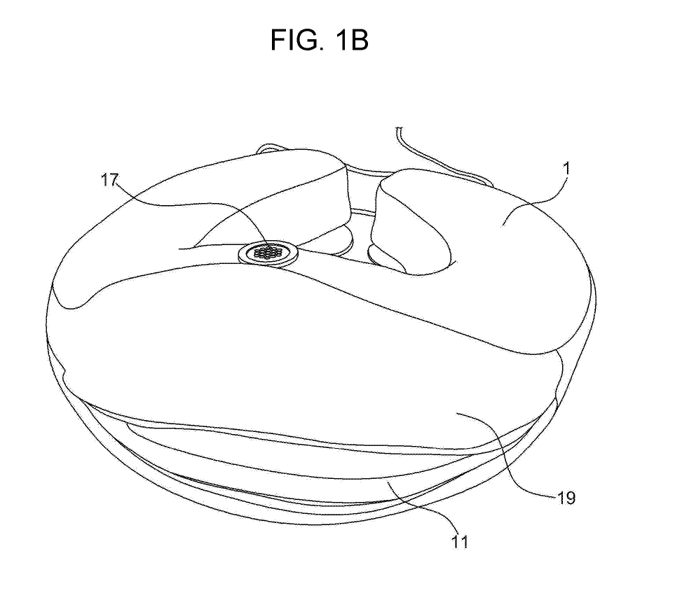

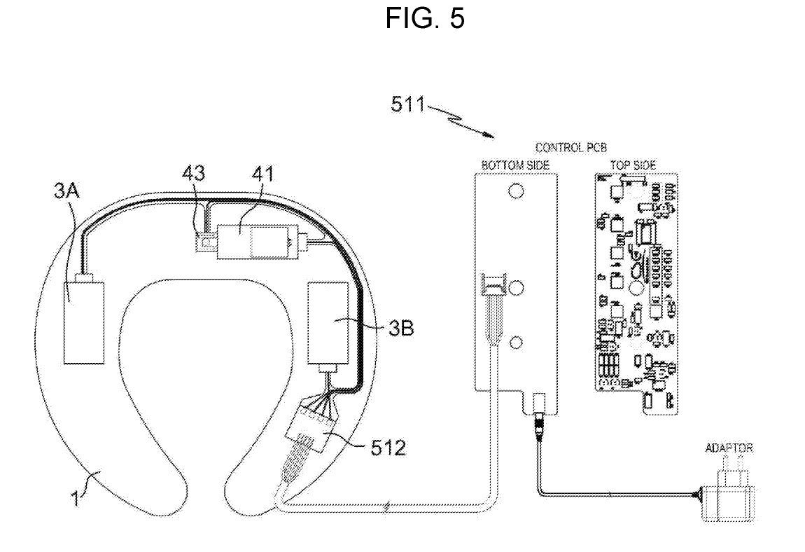

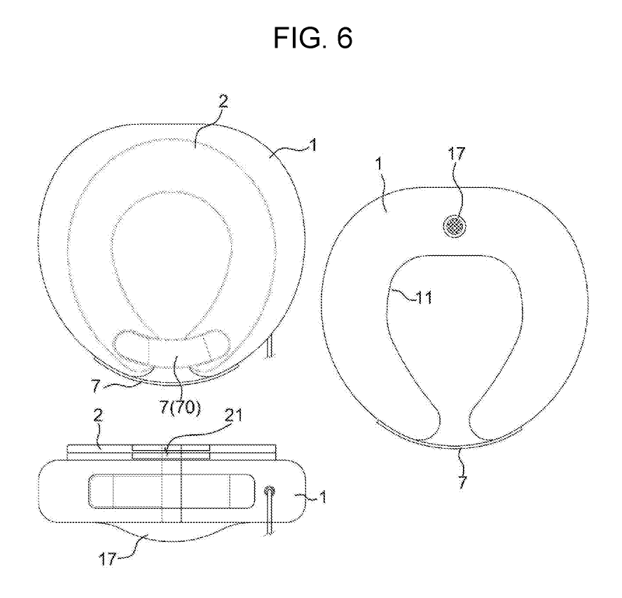

As shown in FIGS. 1 to 7, the neck traction apparatus N according to the present invention includes a main body 1, a traction body 2, a vibration unit 3, an air injection unit 4, a controller 5, a traction assisting device 6, and a fastening member 7.

The main body 1 wraps around the user's neck.

The main body 1 includes a support body 11 and an outer cover 19 covering the outer surface of the support body 11.

The main body 1 has a “U”-like shape having an open front portion, and a user puts on the main body 1 around his/her neck through the open front portion when using the present apparatus N.

The support body 11 is manufactured by foam molding, such that the support body has elasticity sufficient to prevent the user from feeling tenderness upon traction, while maintaining the shape thereof.

The support body 11 has the vibration unit 3 and the air injection unit 4 which are embedded therein. For convenience of embedding the same, the support body 11 is comprised of an upper body 11A and a lower body 11B.

The support body 11 has a first mounting portion and a second mounting portion 15 which are formed to embed the vibration unit 3 and the air injection unit 4 therein.

The first mounting portion 13 has the vibration unit 3 embedded therein and is formed on each of the left and right sides of the support body 11 such that the vibration units 3 are vibrated at the left and right sides in a balanced manner. The second mounting portion 15 has the air injection unit 4 embedded therein and is formed at the rear side of the support body 11.

The second mounting portion 15 of the lower body 11B is has an air passage hole 16 formed therein for intake of outside air and exhaust of intake air. The outer cover covering the support body 11 has an intake and exhaust plate 17 provided at a position corresponding to the air passage hole 16 and having ventilation holes formed therein for air intake and exhaust.

The lower body 11B has a protruding portion 12 formed at a rear lower portion thereof and supported in contact with the bottom of the user's neck upon wearing such that the main body 1 is prevented from shaking forward and rearward.

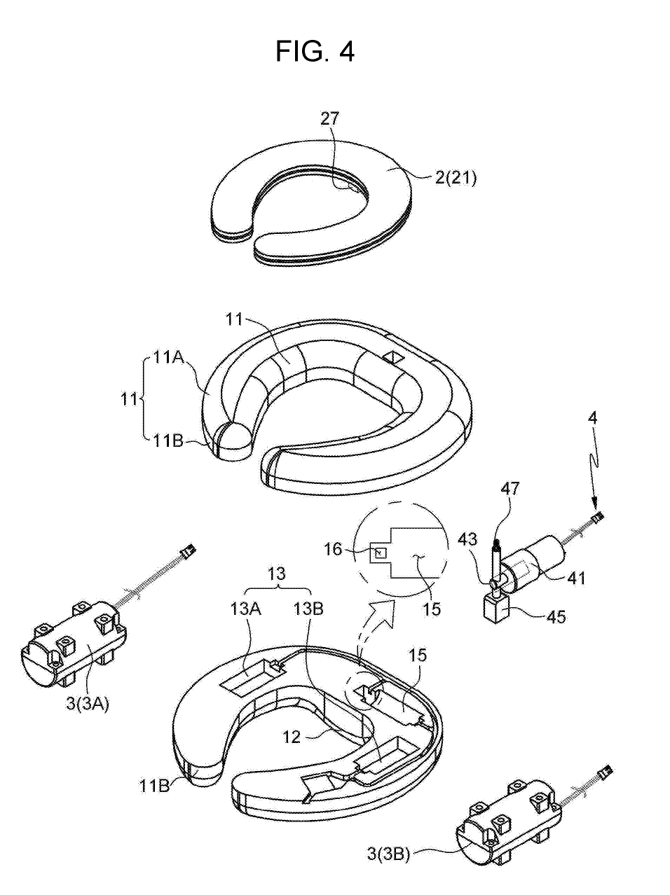

An embodiment relating to the first and second mounting portions 13 and 15 will be described with reference to FIG. 4. The vibration unit 3 and the air injection unit 4 are embedded in the first and second mounting portions 13 and 15 of the lower body 11B, respectively. Thereafter, the upper body 11A is assembled to the lower body 11B to cover an upper portion of the lower body, whereby the vibration unit 3 and the air injection unit 4 are embedded in the first and second mounting portions 13 and 15.

The traction body 2 wraps around the user's neck and is configured to pull and release the user's neck according to the inflation and deflation due to the injection and discharge of air. As shown in FIGS. 4 to 6, the traction body is coupled to an upper portion of the main body 1 and has an open front portion.

The traction body 2 includes a tube 21 which inflates or deflates according to the injection and discharge of air and an outer cover (see FIGS. 1a and 1b) covering an outer surface of the tube 21.

The traction body 2 also has a “U”-like shape having an open front portion, and the user puts on the traction body 2 around his/her neck through the open front portion when using the present apparatus N.

It is preferable that the traction body 2 has a multi-layer structure for the purpose of neck traction. In the drawings, the traction body 2 has a two-layer structure.

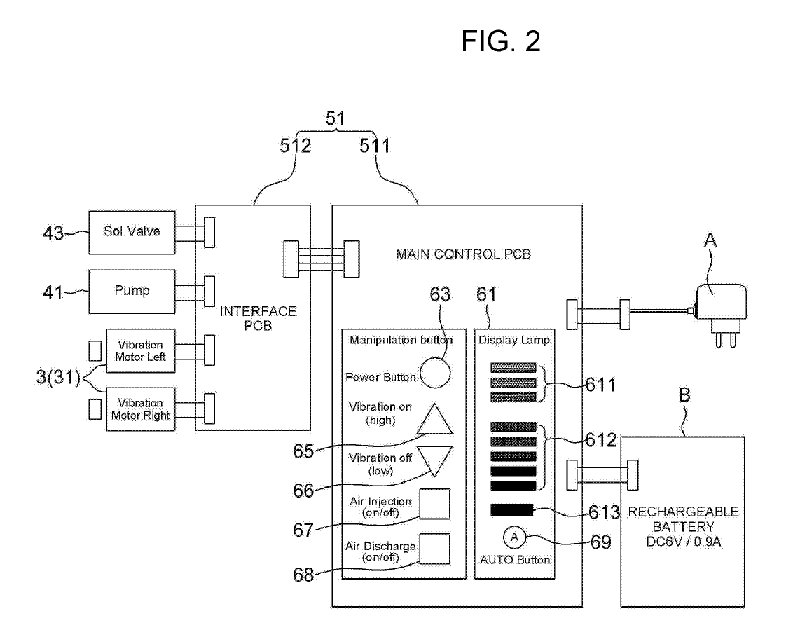

The vibration unit 3 is embedded in the first mounting portion 13 of the main body 1 and is configured to generate vibrations. As shown in FIG. 2, the vibration unit includes a vibration motor 31.

As described above, vibration units 3A and 3B may be provided at the left and right sides of the main body 1, respectively. A detailed description of the structure of the vibration unit 3 is omitted herein, but it is expected that the description described in this specification will not cause any difficulties for those skilled in the art to carry out the present invention.

With the provision of the vibration unit 3, the present invention can further provide a muscular pain relief effect and a massage effect on the neck area through vibrations generated by the vibration unit 3 upon neck traction.

The air injection unit 4 is embedded in the second mounting portion 15 of the main body 1 and is configured to inject air into the traction body 2 and exhaust the air therefrom such that the traction body inflates and deflates thereby. As shown in FIGS. 2, 4, and 5, the air injection unit includes an air pump 41 sucking outside air and discharging the sucked air to the traction body 2, a valve 45 opening or closing an air passage to cause intake and exhaust of air to be performed or stopped, a nozzle coupled to a connector 27 of the tube 21 of the traction body 2 in an attachable and detachable manner, an intake and exhaust induction pipe 43 connecting the valve 45 and the nozzle 47 to the air pump 41 such that intake and exhaust of air are performed.

The neck traction apparatus N according to the present invention is provided with a battery B so as to be carried while in use.

The controller 5 controls the operations of the vibration unit 3 and the air injection unit 4 and charges the battery B by using external power.

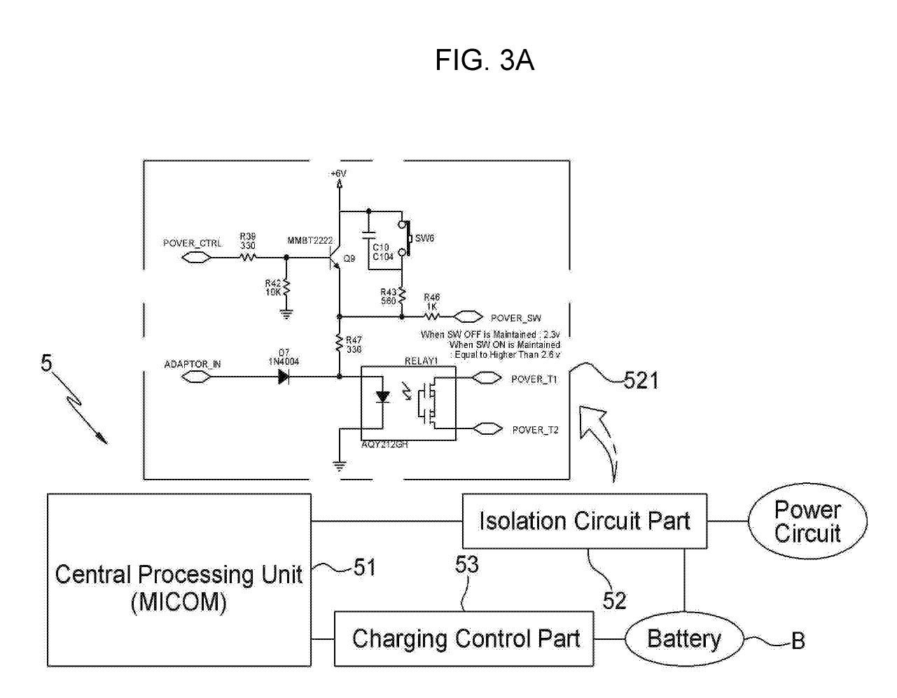

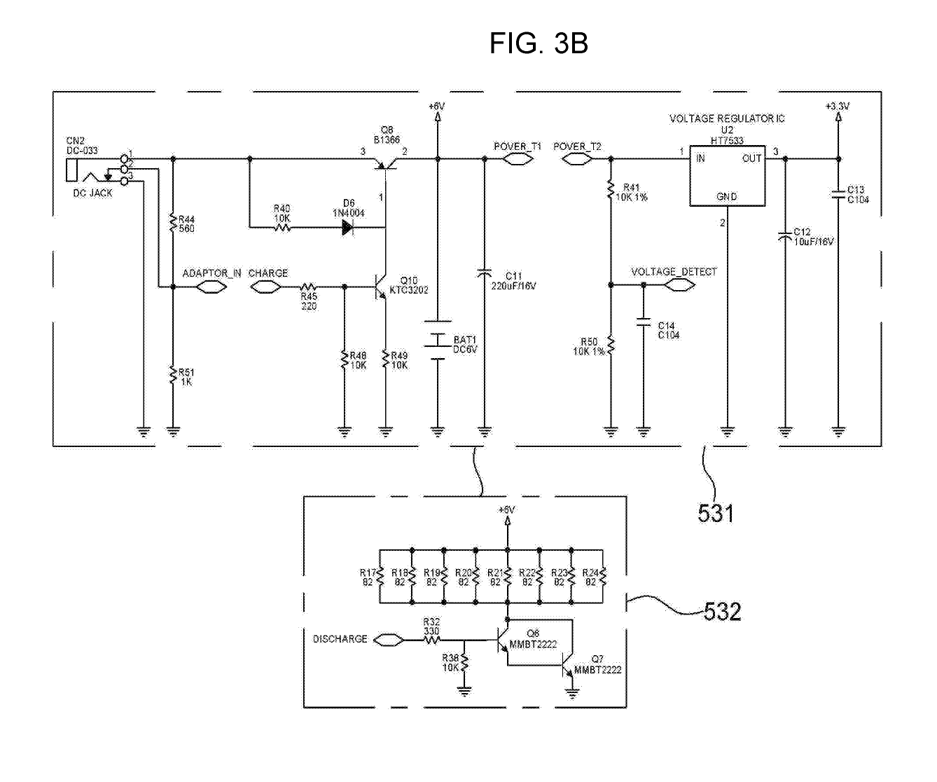

As shown in FIG. 2 and FIGS. 3a and 3b, the controller 5 includes a central control part 51, an isolation circuit part 52, and a charging control part 53.

Each part will be separately explained. First, the central control part 51 is configured to transmit a control signal (control command) to manage overall control of the present apparatus N. For this purpose, it is preferable that there may be included a central processing unit (CPU, MCU, MICOM, etc.).

Furthermore, as shown in FIG. 2, the central control part 51 includes a main control module 511 controlling overall operation of the present apparatus N and an interface module 512 providing a communication port for controlling the vibration unit 3 and the air injection unit 4 (air pump 41 and valve 43).

As shown in FIG. 2 and other drawings, the main control module 511 controls operations, including power on/off, vibration on/off, air injection/discharge, battery capacity display, operation display, auto display, automatic mode execution, and the like. The main control module may mechanically include button members relating to the above-mentioned operations, a light emitting diode (LED) display lamp, an automatic mode execution button, a main body connecting cable, a charging adaptor A connecting portion, and the like.

Next, the isolation circuit part 52 is configured to provide connection or disconnection with external power for charging the battery B. The external power is supplied through the adaptor A connected to a commercial power source providing 220V. It is preferable that the isolation circuit 52 includes a relay circuit 521 shutting off physical and electrical connection with the battery B when external power is not supplied.

As can be seen in FIGS. 3a and 3b, the relay circuit 521 includes a reverse current prevention diode and a power-off relay. The isolation circuit part 52 having the relay circuit 521 physically and electrically isolates the battery B when external power is not applied, thus preventing the battery B from being consumed while in standby and thus minimizing the discharging of the battery B.

Next, the charging control part 53 is configured to charge and discharge the battery B. It is preferable that the charging control part 53 includes a charging circuit 531 and a discharging circuit 532 which are directly controlled by the central control part 51.

As can be seen in FIG. 3b, the charging circuit 531 is composed of a switching transistor, a resistor and a diode for stabilizing the applied voltage, and bias resistors. The discharging circuit 532 is composed of a switching transistor and a discharging load group (resistors configured in parallel with each other).

The charging control part 53 including the charging circuit 531 and the discharging circuit 532 allows the central control part 51 to directly control charging and discharging of the battery B. This makes it possible to omit provision of a separate integrated circuit (IC) involved in charging and discharging of the battery B, thus reducing development cost.

Furthermore, as can be seen in FIG. 3b, the charging control part 53 further includes a constant voltage circuit. Through provision of the constant voltage circuit, it is possible to prevent the overcharging and overdischarging of the battery, thus improving stability and preventing performance deterioration due to the discharging of the battery B.

Meanwhile, there may be a case where the use of the present apparatus N is inconvenient, such as a case where the user's beard touches the skin contact surface of an upper portion of the traction body 2, which makes the user feel irritated or a case where the inflation length of the treatment body 2 is insufficient for a user with a long neck.

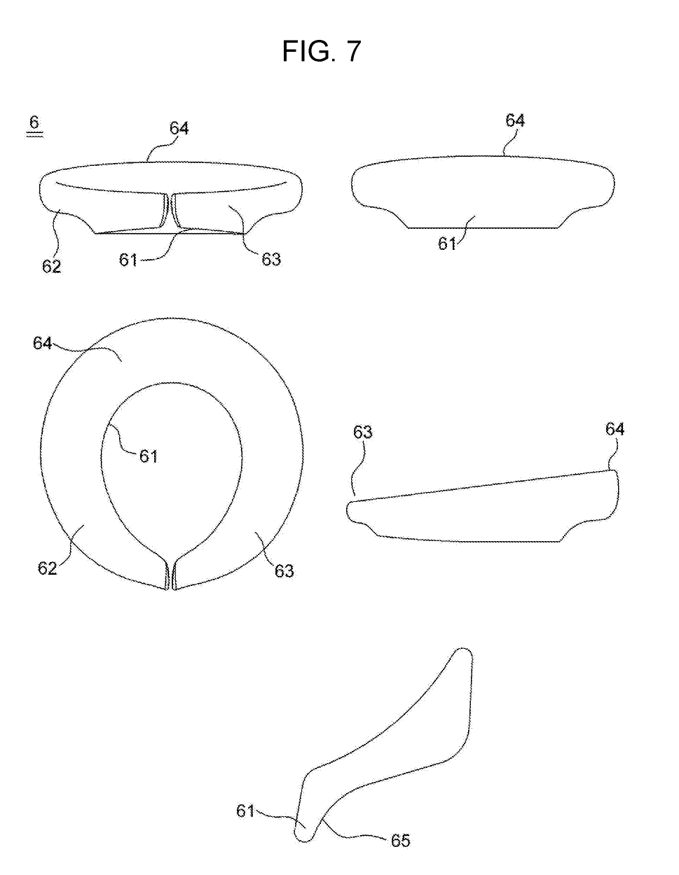

The traction assisting device 6 is configured to eliminate such inconvenience and is seated on the upper portion of the traction body 2 of the present apparatus N as shown in FIGS. 1a and 1b and FIG. 7.

It is preferable that the traction assisting device 6 is made of integral skin foam (ISF) type for a comfortable fit. The ISF is not harmful to the human body and provides soft feeling and cushion while ensuring excellent durability, thus making the user feel a comfortable fit.

As shown in FIG. 7, the traction assisting device 6 also has a “U”-like shape having an open front portion like the main body 1 and traction body 2 and wraps around the user's neck.

The traction assisting device 6 is configured to gradually increase in width and height from a first side portion 62 and a second side portion 63 toward a rear side portion 63 so as to stably support the back of the neck. This makes it possible to improve the neck traction effect, and also to intensively stretch the back of the user's neck, thus being excellent in alleviating “forward head posture” which is a chronic disease of modern people.

The traction assisting device 6 is a configuration which the user may select and thus is not secured to the main body 1 and the traction body 2. This may cause the traction assisting device 6 to be separated from the traction body 2 while in use. If separated, a stretching function of the traction assisting device 6 may be reduced or lost due to the structure in which the front portion thereof is open.

The traction assisting device 6 has a seat portion 61 protruding from a lower portion thereof to be caught by being seated on the upper inside of the traction body 2, such that the traction assisting device 6 is prevented from being separated from the traction body 2 while in use.

Although the seat portion 61 of the traction assisting device 6 is seated and positioned on the inside of the traction body 2, the seat portion 61 may be released from a seated position as the traction body 2 repeatedly inflates and deflates. When the seat portion 61 is released from the seated position, the traction assisting device 6 is loosened at the open front portion, leading to a reduction in effect of the cervical traction.

Accordingly, the seat portion 61 is configured such that a lower surface thereof is inclined to form an inclined surface 65, whereby even when the traction assisting device 6 is separated from the traction body 2 while the traction body 2 deflates, the seat portion 61 of the traction assisting device 6 is seated naturally on the inside of the traction body 2 upon inflation of the traction body 2. This makes it possible to maintain the effect of the cervical traction of the traction body 2.

The fastening member 7 is adapted to secure each of the main body 1 and the traction body 2 such that the open front portion thereof is kept closed upon inflation.

It is preferable that the fastening member 7 is a Velcro 70 having one end secured to one end of each of the main body 1 and the traction body 2 and the other end attached and detached therefrom. Through provision of the fastening member 7, the present invention provides a stable wearing effect.

Hereinafter, the processes of stretching and releasing the neck according to inflation and deflation of the traction body 2 will be described.

Referring to FIG. 2, the controller 5 is provided with an LED display lamp 61 displaying an operation state to inform the user of the operation state for a user's manipulation, and buttons 63 to 68 manipulated by the user.

The LED display lamp 61 displays various operation states of the neck traction apparatus N by using the color of emitted light and on/off and flashing of light.

For example, when the LED display lamp 61 illuminates in three kinds of colors of blue, red, and green, the following various states are displayed.

When power is turned on, an LED unit 611 displays the capacity of the battery as three levels and illuminates in red at the first level and illuminates in green at the second and third levels. The intensity of the vibration unit 3 is displayed by an LED unit 612 such that a blue lamp is turned on additionally from a first level to a fifth level in a stepwise manner. In an automatic mode, an LED unit 613 for an AUTO display green lamp is turned on.

The buttons manipulated by the user include a power button 63 for turning power on and off, up and down buttons 65 and 66 for adjusting the vibration intensity of the vibration unit 3, an injection button 67 for injecting air into the traction body 2, and a discharge button 68 for discharging air from the traction body 2.

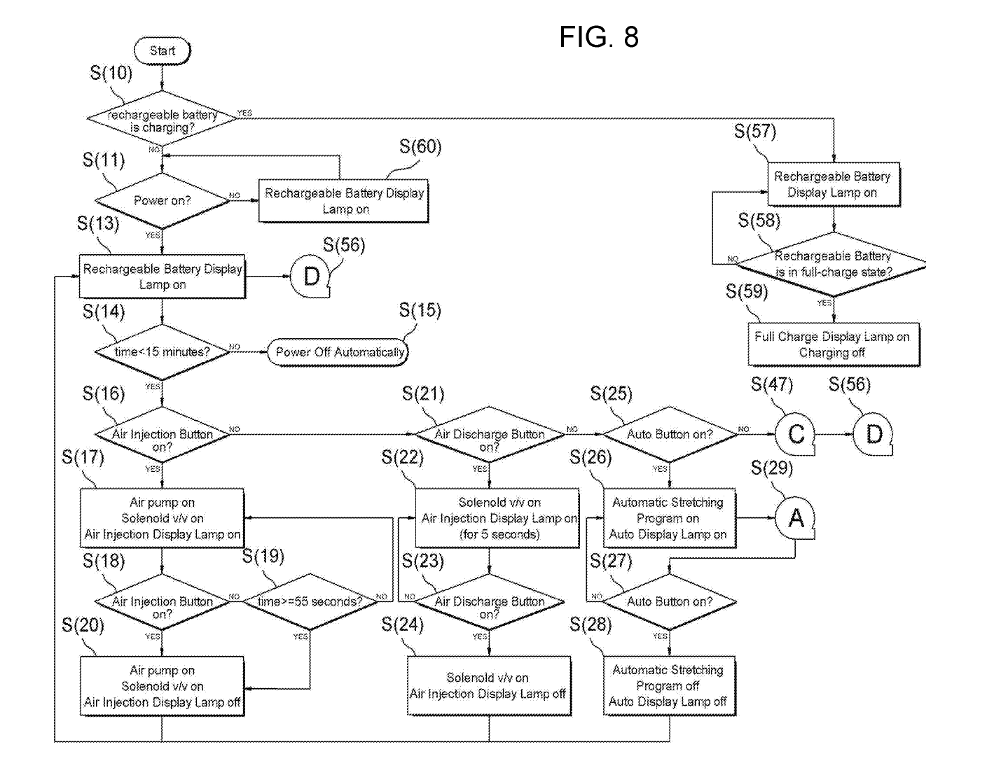

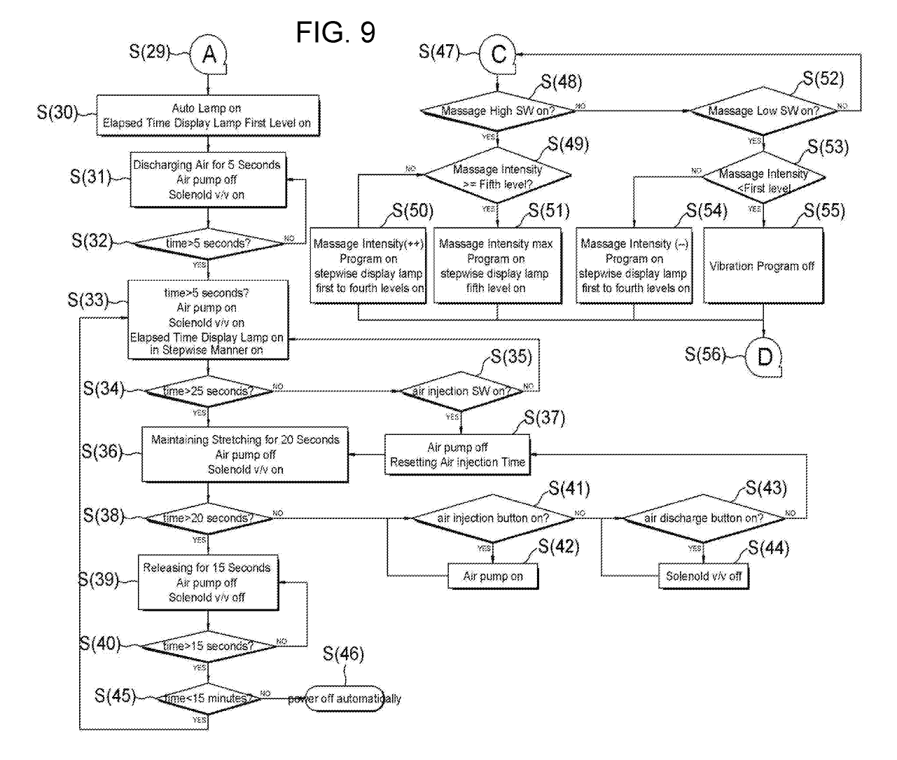

FIGS. 8 and 9 are flowcharts showing operational processes of the neck traction apparatus according to the present invention, which makes it possible to easily understand the operation of the neck traction apparatus according to the present invention.

When the power button 63 of the controller 5 is pressed to turn on power (S11), the LED unit 611 displays the capacity of the battery (S13).

To prevent problems such as a safety accident that occurs after long term use when the user is asleep in a stretching state of the neck, or unnecessary power consumption due to continuous operation of the apparatus after the apparatus is removed from the neck, power is automatically turned off after 15 minutes after the power is turned on (S14 and S15).

When the air injection button 67 is pressed, air is injected into the traction body 2, and then when the air injection button is pressed again, air supply is stopped (S16 to S20).

When the air injection button 67 is not pressed in a state where air is injected by pressing the air injection button 67, air supply is automatically stopped after 55 seconds for safety (S19).

When the air discharge button 68 is pressed, air is discharged and released, and when the air discharge button is pressed again, air discharge is stopped (S21 to S24).

When an auto button 69 is pressed, the LED unit 613 for the AUTO display green lamp is turned on and the automatic mode is entered (S25 to S46).

When the air discharge button 68 is pressed again, the automatic mode is released (S22 and S23).

In the automatic mode, a process of injecting for seconds, maintaining stretching for 20 seconds, and releasing for 15 seconds is repeated for 15 minutes (S45). This process of the automatic mode is significantly effective for the cervical correction of the user.

In the automatic mode, the above-described air injection may be performed on the basis of the set time and may be performed on the basis of the set pressure such that air injection is performed until a pressure sensor senses a predetermined pressure.

In the automatic mode, in a case where air pressure is considered to be insufficient or excessive while the stretching state is maintained, when the air injection button or the air discharge button is pressed, air is injected or discharged while the button is pressed. Herein, adjusted air pressure is stored in a memory and applied to the later stretching (S41 to S44).

When the vibration button is pressed in a state where power is turned on, the apparatus is operated at the first level of the vibration intensity (S48).

When the up and down buttons 65 and 66 are pressed, the vibration intensity is adjusted from the first level to the fifth level (S48 to S55).

Although the preferred embodiment of the neck traction apparatus having the traction assisting device according to the present invention having specific shapes and configurations have been disclosed for illustrative purposes, those skilled in the art will appreciate that various modifications, additions and substitutions are possible, without departing from the scope and spirit of the invention as disclosed in the accompanying claims.

1. A neck traction apparatus having a traction assisting device, the apparatus comprising:
a main body having an open front portion and wrapping around a neck of a user;
a traction body connected to an upper portion of the main body and having an open front portion, the traction body wrapping around the neck of the user, the traction body being configured to inflate to stretch the neck;
a vibration unit embedded in the main body to vibrate the main body;
an air injection unit embedded in the main body and injecting air into the traction body to inflate the traction body;
a traction assisting device placed on an upper portion of the traction body and wrapping the neck, the traction assisting device having an open front portion, the traction assisting device being configured to gradually increase in height and width from a front side to a rear side thereof.

2. The apparatus of claim 1, wherein the traction assisting device has a seat portion formed at a lower portion thereof, the seat portion being seated and positioned on an upper inside of the traction body, and
the seat portion has a lower surface inclined toward the upper inside of the traction body that inflates.

3. The apparatus of claim 1, wherein the main body includes:
a support body formed by foam molding; and
an outer cover covering the support body, wherein
the support body has a first mounting portion provided at each left and right side thereof and in which the vibration unit is embedded, and a second mounting portion provided at a rear side thereof and in which the air injection unit is embedded.

4. The apparatus of claim 1, further comprising:
a controller including a battery supplying power for driving, an isolation circuit part providing connection or disconnection with external power for charging the battery, a charging control part managing charging and discharging of the battery, and a central control part controlling the charging control part, the vibration unit, and the air injection unit.

5. The apparatus of claim 4, wherein the controller operates the air injection unit in an automatic mode in which a process of injecting air into the traction body for 20 seconds, maintaining stretching for 20 seconds, and then releasing by discharging the air from the traction body for 15 seconds is repeated for 15 minutes.
